(19) United States
(12) Patent Application Publication
(10) Pub. No.: US 2011/0201856 A1
(43) Pub. Date: Aug. 18, 2011

(54) FILTRATION AND PREDISTRIBUTION DEVICE FOR A FIXED CATALYTIC BED REACTOR AND USE THEREOF

(75) Inventors: Bernard Cottard, Saint Romain de Colbosc (FR); Matthew Allen, Orange, TX (US)

(73) Assignee: TOTAL RAFFINAGE MARKETING, Puteaux (FR)

(21) Appl. No.: 13/000,577

(22) PCT Filed: May 26, 2009
(86) PCT No.: PCT/FR09/50967

§ 371 Date: May 6, 2011

(30) Foreign Application Priority Data
Jun. 23, 2008 (FR) FR 08 03491

(51) Int. Cl. C07C 5/00; B01J 8/00; B01D 29/00

(52) U.S. Cl. 585/250; 422/187; 210/456; 210/232

(57) ABSTRACT

The invention relates to a filtration and predistribution device (4) comprising: a flat plate (5) perforated by holes, each hole being overhung by a vertical hollow duct (6) that includes at least one slot passing through the lateral wall thereof, a filtration bed (7) placed on the plate surrounding said ducts, comprising at least one layer of hollow filtering elements (8) that are larger in size than the slots of the ducts, said filtering element being obtained by winding, in touching and/or non-touching turns, a wire of cross section (s) so as to have at least one closed end and having a [free area (S_{free}) of the element/area (Sₘ) occupied by the wire] ratio of between 2 and 50%. The invention also relates to the use of this device (4) for filtering and predistributing at least one particle-laden liquid upstream of a fixed catalytic bed (12) of a reactor.

### TECHNICAL FIELD

The present invention relates to the field of fixed bed catalytic reactors fed with liquid and/or gaseous fluids, able to operate with co-current down-flow or upflow, or alternatively on a countercurrent basis. The invention proposes a novel device, situated upstream of the catalytic bed, capable of improving the filtration of the impurities-loaded stock fluids and the distribution thereof so as to limit the fouling of the surface layers of the catalytic bed.

Hereinafter and within the meaning of the invention, the term “fluid(s)” means liquids and gases and the term “particle(s)” means all of the solid and liquid impurities contained in the fluids. Likewise, and unless indicated otherwise, the expression “comprised between a value X and a value Y” means a range in which the end points X and Y are included.

In the case of trickle bed catalytic reactors which employ co-current down-flow of gas and liquid over a fixed bed of catalyst, correct reactor operation essentially relies on managing the fill of catalyst, the distribution of the phases and the pressure drop across the catalytic bed. The problems caused by poor distribution of the fluids and an increase in pressure drop are essentially connected with the presence of contaminating and plugging particles of various kinds the size of which can vary from 1 μm to 200 μm and which are contained in the stock fluids. Within the field of refining, the particles present in fluids of the hydrocarbons type may be catalyst fines originating from catalytic cracking units of the FCC (Fluid Catalytic Cracking) type and the dimensions of which vary from 5 to 20 μm, particles of corrosion, also known as “rust scales”, originating from the storage facilities and metallic units situated upstream of the reactor, or else coking particles originating from the exchangers. By being deposited on or between the grains of catalyst, these particles can quickly increase the pressure drop across the catalytic bed and greatly reduce the performance of the reactor. The invention aims to resolve the problems associated with the distribution of the fluids and with the pressure drop by proposing a new device for filtering and distributing the stock fluids. Said device of the invention notably aims greatly to reduce the fouling of the catalytic bed by applying an effective filtration of the stock fluids upstream of the catalyst.

In general, in a trickle bed reactor, the process begins with the simultaneous distribution of the stock fluids in the top of the reactor. Even from this early stage, the quality of the distribution of the liquid toward the catalytic bed is of key importance. For that reason, liquid has to be distributed as a fine and even rain. To do this, and according to one preferred configuration of the reactor, the particle-laden liquid is dispersed in the form of homogeneous and multidirectional jets by the stock diffuser and is then split up as it passes through a holed distributor plate (predistribution plate perforated with 40 to 100 orifices per square meter of cross sectional area of catalytic bed). The problem encountered here is connected with the fact that the contaminating particles contained in the liquid may block the orifices in the plate and give rise to defective distribution of the liquid phase. The fluids then reach a perforated distribution plate or distributor plate which supports chimneys. This plate allows the liquid and the gas to mix inside the chimneys (the semi-open upper end of the chimneys allows the reactive gas to pass while apertures situated in the bottom part allow the liquid to pass). On leaving the chimney, the diphasic mixture passes, by a plug flow, through several 10 to 15 cm thick layers of solid inert beads made of silica and alumina. From the upstream end downstream, these layers are usually distributed in a gradient of decreasing particle size. These inert beads have the task of dividing the charge stock and of redistributing it to avoid creating preferred circuits, which are the sources of hot spots and of coking in the catalytic bed. Below the inert beads and stabilized by these, the catalytic bed can extend over a height of 5 to 10 m. Depending on the path taken by the liquid, the small-sized contaminating particles pass through the layers of inert beads and accumulate on the surface of the catalytic bed. This causes progressive obstruction of the free gap zones situated between the grains of catalyst. This progressive fouling of the layers of the catalytic bed may have the effects of gradually increasing the pressure drop across the catalytic bed and of blocking the mixing chimneys, thereafter causing deformation of the distribution plate and incorrect distribution of the fluids across the catalytic bed. It may then become necessary to shut down the unit prematurely so as to change all or part of the catalyst, and to do so even before the catalyst has completely lost its catalytic activity. The frequency of interventions may vary wildly. Usually, shutdowns are performed every 12 to 18 months so as to reload with new catalyst and new beads. However, it is sometimes necessary to carry out catalyst descaling operations every 2 to 3 months. Each of these unit shutdowns for interventions on the catalytic bed (scaling or replacing the catalyst) has a considerable financial impact. It would therefore appear to be essential to avoid these and to seek to prolong the activity of the catalyst significantly. The invention seeks to meet these requirements by proposing a new device for the filtration and distribution of fluids which is situated upstream of the distribution plate.

### PRIOR ART

Numerous devices have already been proposed for improving the filtration of the liquid charge and the distribution thereof. The technologies most representative of the field under study are described below:

U.S. Pat. No. 3,992,282 proposes the use of “trash baskets” that pass successively through a layer of inert beads and then through the catalytic bed. When the upper part of the catalytic bed is fouled, the charge bypasses the crust of scales formed at the surface of the catalyst by taking a path through the baskets. The mesh that constitutes the lower half of the basket allows the charge to emerge into the lower layers of the catalyst, trapping the particles or scales contained in it. This device does, however, have some disadvantages:
The mesh portion of the basket may quickly become obstructed by the impurities in the charge and as a result, the lifecycle of the catalytic bed is not lengthened significantly.
The distribution of the fluids may be disrupted if the “trash baskets” are not distributed evenly and positioned vertically. Such a situation appears difficult to obtain.

U.S. Pat. No. 4,313,908 describes a reactor in which an auxiliary catalytic bed is inserted downstream of a distributor plate supporting chimneys and upstream of the main catalytic bed. Tubes of two different lengths, arranged in an alternating pattern, pass right through the auxiliary bed. When the auxiliary bed is fouled, the liquid takes the path of least resistance. Thus, the liquid charge passes through the short-length tubes by overspilling while the gas continues to pass along the longer-length tubes. This device makes it possible to bypass the crust of scales on the auxiliary catalytic bed and reach the main catalytic bed. The length of activity of the reactor is thereby prolonged. This device does, however, have some disadvantages. Specifically, in order to implement this technology, it is necessary to increase the dimensions of the reactor so as to keep the same volume of catalyst in the main bed and install a new catalyst support grating, which entails carrying out welding operations inside the reactor. In addition, it is not easy to control the flow rates of gas and liquid during the reaction because the path taken by the liquid differs between the start and end of operation, unlike that of the gas.

Like the aforementioned patent, patent EP1200183 proposes devices aimed at reducing the pressure drop by altering the path taken by the fluids. This patent notably relates to a bypass device inserted within the catalytic bed. This device consists of a first tubular cage element containing, at its center, a second hollow elongate element intended to receive the charge as soon as the upper layer of the catalytic bed becomes fouled. The charge stock is thus distributed to the lower layers of the catalytic bed with no significant pressure drop. However, this solution is not optimal because, like the previous ones, it occupies a not-inconsiderable volume of catalytic bed, thereby accordingly reducing the ability of the latter to react with the charge.

Patent application FR 2 229 759 proposes filtration devices fixed on a plate situated upstream of the catalytic bed. Such a filtration unit may consist of two cylinders which are coaxial with one another and with the reactor. The inner cylinder is closed at its top and open at its bottom, while this configuration is reversed in the case of the outer cylinder. The walls of the cylinders are perforated and the chamber between the cylinders may contain catalytic material identical to or different than that of the catalytic bed. The charge containing the particles enters the outer cylinder where it is filtered, then reemerges via the open bottom of the inner cylinder to come into contact with the catalytic bed. The disadvantage with this device lies in the fact that the filter cartridges may become fouled, potentially leading to the plate becoming completely bunged up and the reactor having to be shut down.

Finally, patent FR 2 889 973 discloses a device for the filtration and distribution of the gaseous and liquid phases which consists of a perforated plate situated upstream of the catalytic bed and to which mixing chimneys are attached. A filtration bed, consisting of various layers of particles, is supported by the plate and surrounds the chimneys. Each chimney may be separated from the filtration bed by means of a grating the size of mesh of which is smaller than that of the particles of the filtration bed. Within the meaning of that patent, the particles making up the catalytic bed are inert particles formed of silica or of alumina, particles that are active with respect to the chemical reaction taking place on the catalytic bed or alternatively bits of structured packing. The filtration bed consists of at least one layer of particles of a size smaller than or equal to the size of the particles of the catalytic bed. The gas enters the chimneys via the top openings while the liquid passes through the filtration bed and then enters the chimneys via lateral slots. The filtration bed gradually becomes clogged starting with the lower layers and has to be replaced at least every six months. The effectiveness of this device may be limited by the partial or complete blockage of the circular gratings and then of the chimneys causing poor distribution of the liquid across the remainder of the chimneys that remain open and causing an increase in the pressure drop. In addition, the presence of orifices in the plate does not encourage mixing between the gaseous and liquid phases within the chimney because the liquid is able to pass through the filtration bed and then leave by the orifices in the plate without entering the chimney in which the gas is circulating.

U.S. Pat. No. 3,584,685 describes a tubular filtration element supported by a support plate. This filtration element is formed of a helical wire fixed to rods attached to the plate at right angles to the latter and is therefore secured to the plate, its axis being perpendicular to the surface of the plate.

However, in that document, the filtration element is secured to the plate and at no time is any other use of this element, notably the “bulk” use thereof in a filtration bed, imagined.

The present invention sets out to solve the problems encountered in the prior art. The invention therefore proposes a novel device for the filtration and distribution of stock fluids, capable of reducing the fouling of the upper layers of the catalytic bed with a view to prolonging the activity of the catalyst. The main advantage of said device is that it maximizes the useful volume of the catalytic bed by being positioned upstream thereof, preferably upstream of the distributor plate. In this regard, and by comparison with the distributor plate, the device of the invention will hereinafter be termed fluid “predistribution” device. The other advantages of the invention will be revealed by the examples.

### DESCRIPTION OF THE INVENTION

The invention relates to a device for the filtration and predistribution of at least one particle-laden fluid fed to a reactor containing at least one fixed catalytic bed. Said fixed bed(s) catalytic reactor may be fed with liquid and/or gaseous fluids and operate on co-current down-flow or up-flow or alternatively on a counter-current basis. In such a reactor, the device of the invention is situated upstream of the catalytic bed, preferably upstream of the distributor plate which may serve to support mixing chimneys. In one embodiment in which the reactor has just one fixed catalytic bed, the device of the invention is positioned in the empty space situated between the fluid(s) diffuser and the distributor plate. In another embodiment in which the reactor comprises more than one fixed catalytic bed, there may be as many devices as there are catalytic beds. In this configuration, each additional device according to the invention is preferably positioned between the quench box which cools the reactor by quenching and the holed splitter plate situated downstream or, failing that, between the quench box and the distributor plate.

In this application, the terms “upstream” and “downstream” are to be understood with reference to a down-flow through the reactor.

The device of the invention comprises:
a flat plate perforated with orifices, which is intended to be positioned parallel to the transverse cross section of the reactor. Each orifice in the plate lies in vertical alignment below a vertical hollow chimney comprising at least one aperture passing right through its side wall;
a filtration bed arranged on the perforated plate and surrounding said chimneys, the filtration bed comprising at least one layer of hollow filtering elements the dimensions of which are greater than the dimensions of the apertures of the chimneys, each filtering element being obtained by winding a wire of cross section (s) into contiguous and/or non-contiguous turns so as to have at least one closed end, and having a ratio of the free surface area (S_{free}) of the element to the surface area (S_{wire}) occupied by the wire that is comprised between 2 and 50%.

Advantageously, each chimney is removable. Depending on the properties of the fluid to be treated and on the size of the filtering elements chosen for purifying said fluid, it is then possible to fit to the flat perforated predistribution plate chimneys the dimensions of the aperture or apertures of which are smaller than the smallest dimension of the filtering elements surrounding it, so that these filtering elements cannot enter the chimney via the aperture.

Advantageously, said at least one aperture of each chimney extends in a substantially helical path along the side wall of the chimney.

The axis of this helical path therefore coincides with the vertical axis of the chimney, it being possible for the pitch of this path to be variable. This aperture may run continuously or discontinuously along the path. Producing chimneys each of which is provided with one continuous aperture over substantially the entire height of the chimney has the advantage of encouraging the gas to circulate through the chimney and prevent it from becoming clogged. In addition, such a chimney can easily be defouled by vibration, for example by the vibrations induced by bending the chimney over or stretching it and then releasing it.

The orifices in the perforated flat predistribution plate are uniformly arranged so that they have a distribution density comprised between 5 and 150 orifices per m² of plate surface area, preferably of between 30 and 100 orifices per m² of plate surface area.

The perforated flat plate of the device is preferably situated in place of the standard predistribution plate and therefore rests on the support beams that already exist inside the reactor. The perforated flat plate of the device therefore, in terms of its shape and its dimensions, matches the internal transverse cross section of the reactor.

With preference, and regardless of its geometry, each chimney is obtained by winding a wire of cross section (s′) into non-contiguous turns of constant pitch over its entire height, this height for example being comprised between 100 and 1500 mm, preferably between 150 and 600 mm.

In this case of a chimney obtained by winding a wire into turns, the pitch of the turn will then be chosen to be smaller than the smallest dimension of the filtering elements, possibly combined with other elements, which surround it.

As an alternative, the pitch of the turns may vary along the height of the chimney, zones of contiguous turns alternating, for example, with zones of non-contiguous turns.

Because the winding of the wire that makes up the chimney can be likened to that of a spring, it is possible to give it any geometry, for example to make it cylindrical, spherical, barrel-shaped, amphora-shaped, conical, oblong, square, polygonal and any cross section, for example a round, square, rectangular, triangular, oval, etc. cross section. The path of the aperture or apertures may, depending on the geometry of the chimney, not be in the form of a regular helix.

With preference, the chimneys according to the invention are cylindrical and their lateral apertures describe a helix the pitch of which may be variable.

In one preferred embodiment of the invention, the chimneys are in the shape of a cylinder with an inside diameter Di′ at least equal to that of a circular orifice of the perforated plate, of a total height comprised between 100 and 1500 mm and the helical aperture of which has a constant pitch over the entire height of the chimney.

The preferred parameters of such a chimney are as follows:
Height: between 150 and 600 mm, preferably equal to 300 mm.
Inside diameter: between 20 and 500 mm, preferably equal to 60 mm.
Cylinder material: any material capable of withstanding the pressure and temperature stresses of the reactor, preferably stainless steel of the INOX 321. 316L or 304 type.
When the chimney is obtained by winding a wire (F′) of cross section (s′) into non-contiguous turns of constant pitch over the entire height, then the pitch of the non-contiguous turn is less than 50 mm, preferably less than 20 mm.
Diameter of the wire used to form the cylinder: between 5 and 15 mm.

For preference, each chimney is in the form of a cylinder and has open ends at least one of which terminates in a radial return (lug) of the wire of cross section (s′) of a length comprised between ⅓ and ⅔ of the diameter of the cylinder.

Advantageously, at least one end of the chimney is shaped so that it can be fitted by hand and reversibly onto a cylindrical cuff. For preference, this cuff can be secured to an orifice in said perforated plate, allowing the chimney to be easily mounted on and removed from the perforated plate. For preference, both ends of the chimney are shaped in this way.

When the chimney is provided with a radial return, the cuff is, for example, provided with a notch the geometry of which allows assembly of the male/female type with the radial return.

Advantageously, one of the ends of the chimney is provided with a cuff inserted in an orifice in the perforated plate, and the other end of the chimney is provided with a cuff covered by a capping element.

The chimneys inserted on the perforated flat plate are preferably identical to one another in terms of dimensions and in terms of shape.

The chimney is made of any material capable of withstanding the extreme pressure, temperature and corrosion conditions of industrial processes, such as metallic materials (steel, stainless steel, bronze, beryllium bronze, etc.), alloys (“Monel”, “Inconel”, etc.), ceramic, plastic (polypropylene, PVDF, C-PVC, PFA, ETFE, ECTFE, PTFE, etc.), composites, graphite, glass. For preference, the chimney is made of stainless steel or steel.

According to the invention, each chimney of the device is surrounded by a filtration bed. Each chimney protrudes for example beyond the level of the filtration bed by a height comprised between 20 and 70 mm, preferably between 30 and 60 mm.

Advantageously, the total height of the filtration bed is comprised between 100 and 500 mm.

The filtering element is obtained by winding a wire of cross section (s) with contiguous and/or non-contiguous turns so that it has at least one closed end and a ratio of the free surface area (S_{free}) of the element to the surface area (S_{wire}) occupied by the wire that is comprised between 2 and 50%, preferably between 5 and 30%, more preferably still between 15 and 25%.

What is meant by the surface area (S_{wire}) occupied by the wire is the surface area occupied by the wire when the hollow element is developed, over its entire periphery, onto a plane positioned at right angles to the axis of winding of its turns, the free surface area (S_{free}) then corresponding to the surface area not occupied by the wire in this projection. Put differently, the surface area (S_{wire}) occupied by the wire is the surface area of the wire projected onto a surface enveloping the outside of the hollow element concerned, this surface then being opened out and “flattened” onto a plane to make it possible to measure it, the free surface area (S_{free}) then corresponding to the area not occupied by the projection of the wire.

With preference, the hollow element is obtained by winding a single wire into contiguous and/or non-contiguous turns.

Because the winding of a hollow element according to the invention can be likened to that of a spring, it is possible to give it any geometry, for example to make it cylindrical, spherical, barrel-shaped, amphora-shaped, conical, oblong, square, polygonal and any cross section, for example a round, square, rectangular, triangular, oval, etc. cross section.

For preference, the filtering element is in the shape of a cylinder or in the shape of a sphere, it being possible for this sphere to be a perfect sphere or one that is slightly deformed depending on the pitch of the turns of the winding.

When the filtering element is in the form of a cylinder, its height is less than or equal to 50 mm, preferably comprised between 10 and 35 mm.

When the filtering element is in the form of a sphere, its inside diameter is less than or equal to 50 mm, preferably comprised between 10 and 35 mm.

Whatever its geometry, the filtering element has two ends, at least one of which is closed. For preference, the filtering element has one open end and one closed end, however, the two ends could be closed, the turns then being non-contiguous.

The closed end of the element may be obtained by winding a wire of cross section (s) in contiguous turns either by winding flat or with narrowing, preferably of the conical type. The element may also be closed off at one of its ends at least by any other capping element, be it flat or three-dimensional, of any appropriate geometry and material.

The filtering element is made of any material capable of withstanding the extreme pressure, temperature and corrosion conditions of industrial processes, such as metallic materials (steel, stainless steel, bronze, beryllium bronze, etc.), alloys (“Monel”, “Inconel”, etc.), ceramic, plastic (polypropylene, PVDF, C-PVC, PFA, ETFE, ECTFE, PTFE, etc.), composites, graphite, glass. For preference, the hollow element is made of stainless steel or steel.

Whatever its geometry, the filtering element may consist, over its entire height, of non-contiguous turns of constant or variable pitch or of contiguous turns or alternatively of a combination of contiguous turns and of non-contiguous turns.

For preference, the filtering element comprises an open end followed by a fluid inlet zone Z1 consisting of non-contiguous turns of pitch P1, followed by a fluid filtration zone Z2 consisting of non-contiguous turns of pitch P2<P1 and which is extended by a closed end of the element. The open end, the inlet zone and the filtration zone may follow on directly from one another or alternatively may be separated from one another by at least one contiguous turn. For preference, the ratio P1/P2 of the pitches of the non-contiguous turns is such that P1/P2≦50, more preferably still, P1/P2≦15. Although the dimensions can be chosen at will, according to the field of application, the filtering element is preferably designed to filter particles the size of which varies from 1 μm to 20 mm.

As explained before, the filtering elements may constitute a filtration bed comprising at least one layer of said elements.

In one and the same layer, the hollow filtering elements are preferably identical to one another, notably in terms of shape and dimensions.

When the filtration bed comprises several layers, these layers are preferably organized in a gradient graded on filtering element size and, more particularly, from the upstream end of the reactor downstream, on a decreasing gradient.

Said hollow filtering elements may be used alone or in combination with other elements, notably of different shapes and/or dimensions and/or functions.

The filtering elements of the device of the invention may notably be combined with other elements, porous or non-porous, such as the inert bodies conventionally used in reactors to improve the diffusion of fluids (for example the inert beads). The elements combined with the filtering elements may also be porous ceramic elements, bits of packing of the Rashig ring, Pall ring type or parts in the form of tiles, elements with a high void fraction and/or particles of catalyst.

When the filtering elements are combined with particles of catalyst these may be identical to or different than those that form the catalytic bed situated downstream. For preference, the elements combined with the filtering elements are particles of pretreatment catalyst capable of trapping the metals contained in the fluid that is to be purified.

The invention further relates to the use of said device in a reactor comprising at least one fixed catalytic bed, the reactor being fed with at least one particle-laden liquid and one reactive gas, said device being situated upstream of the catalytic bed, the perforated flat plate being parallel to the transverse cross section of the reactor.

The liquid and the gas may be a co-current up-flow or down-flow or may be a countercurrent flow.

Advantageously, said device is then positioned upstream of the distributor plate that can support mixing chimneys, which is itself situated upstream of the fixed catalytic bed.

For preference, the device according to the invention is inserted in a reactor for reactions of hydrotreatment, selective hydrogenation, or conversion of hydrocarbon-containing residues or cuts.

The invention is now described with reference to the accompanying nonlimiting drawings in which:

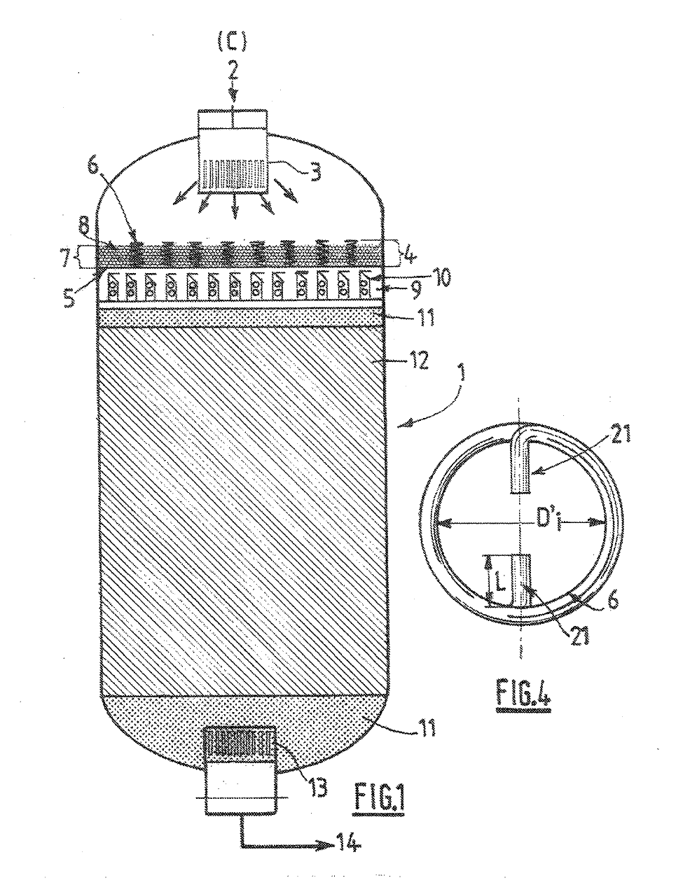

FIG. 1 is a view in longitudinal section of a reactor equipped with a device according to the invention;

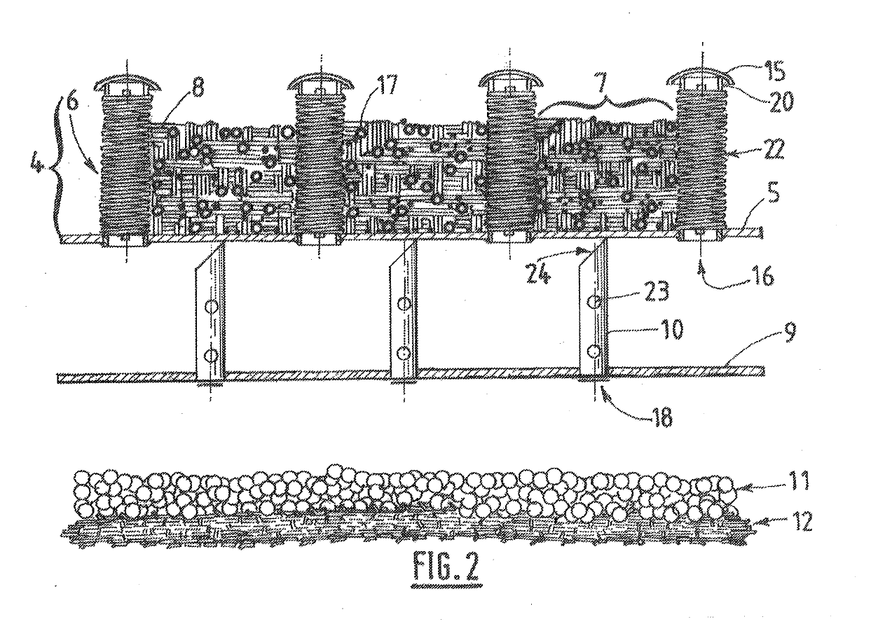

FIG. 2 depicts a view in longitudinal section through the reactor of FIG. 1 showing, in greater detail, the device according to the invention, a distribution plate and the upper part of the catalytic bed;

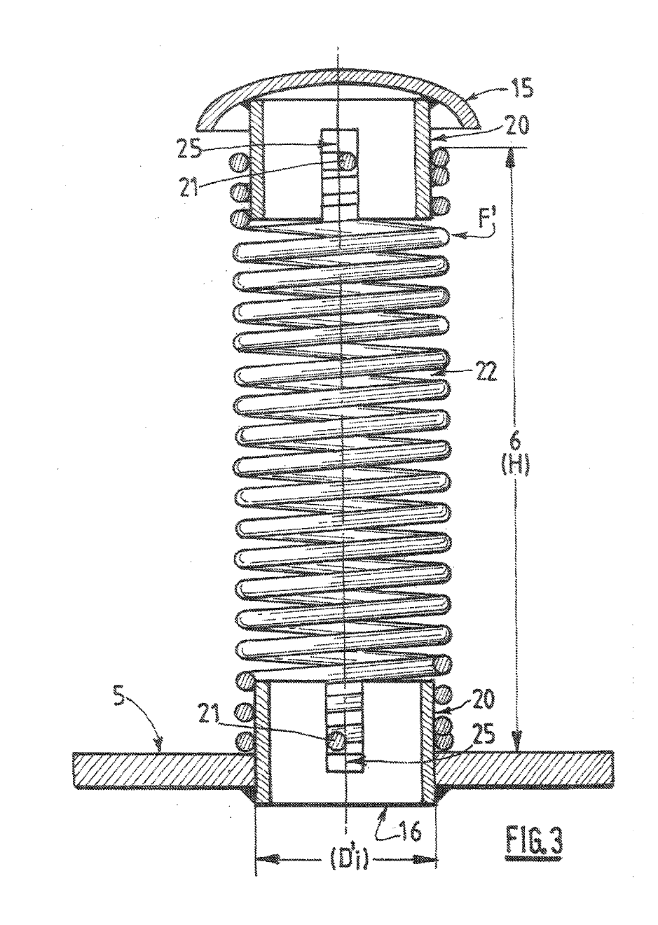

FIG. 3 is a partially sectioned side view of a chimney of the device according to the invention depicted in FIGS. 1 and 2;

FIG. 4 is a view from above of one embodiment of a chimney of the device according to the invention;

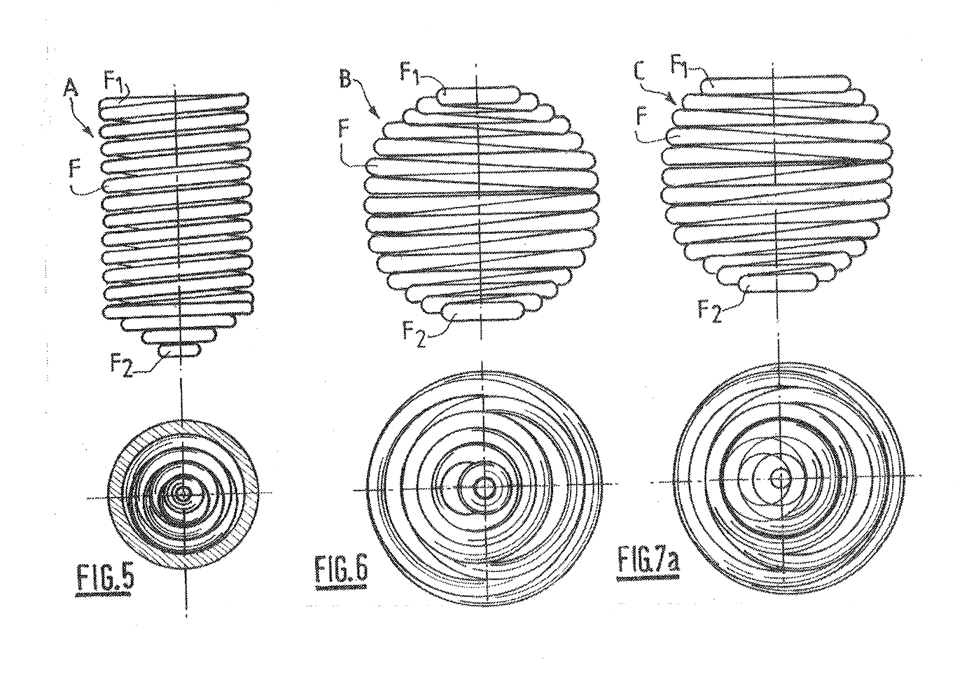

FIGS. 5 to 8 depict exemplary embodiments of filtering elements of the device according to the invention. Each element is depicted in side view and in plan view. The element depicted in FIG. 5 is also depicted in transverse cross section;

FIGS. 9 to 19 depict inert elements mentioned in the examples, viewed from above and in longitudinal section. The dimensions of these elements, in millimeters, are marked on the figures.

The device that was the subject of the present invention is inserted, for example, in a reactor (1) of the kind depicted in FIG. 1, comprising at least one fixed catalytic bed (12) fed with at least one particle-laden fluid (C). In a preferred embodiment, said reactor (1) is fed with a liquid charge and a reactive gas in a co-current downflow. Depending on the configuration of the reactor (1), the liquid and the reactant gas may be introduced simultaneously at the top of the reactor using a charge diffuser (3) or alternatively may be introduced separately, it then being possible for the gas to be introduced at the side of the reactor level with the mixing chimneys (10). Whatever the mode of supply, said fluids (C) are distributed in homogeneous and multidirectional jets directed toward the filtration and predistribution device (4) of the invention.

FIG. 1 depicts a longitudinal section through a fixed catalytic bed reactor (1) fed with a charge stock (C) consisting of liquid and gas in a co-current downflow. The fluids (C) are introduced at the top (2) of the reactor and are dispersed in the form of homogeneous and multidirectional jets by a charge diffuser (3) toward a filtration and predistribution device according to the invention (4).

The latter device comprises a flat plate (5) perforated with orifices (16), the plate serving to support hollow chimneys (6) around which is arranged a filtration bed (7) consisting of at least one layer of filtering elements (8) (FIG. 2).

Each chimney (6) comprises a lateral aperture (22), its top end being provided with a capping element (15), as described in detail hereinafter with reference to FIGS. 2 and 3.

The reactive gas enters each chimney (6) via the end covered with a capping element (15), while the liquid passes through the filtration bed (7). Throughout the path followed by the liquid, the particles (17) contained therein are trapped by the filtering elements (8), inside the filtering elements and in the empty spaces between the elements. The filtered liquid then enters each chimney (6) via the lateral apertures (22). Gas and purified liquid leave the chimney (6) via its open end on an orifice (16) of the perforated flat predistribution plate (5).

The purified fluids (C) leaving the device according to the invention are thus dispersed toward a distribution plate (9) serving to support mixing chimneys (10). In the example depicted in FIGS. 1 and 2, these mixing chimneys (10) are positioned in a staggered configuration in relation to the orifices (16) in the upstream perforated flat plate (5). Here, the gas enters the mixing chimney (10) via its semi-open upper end (24) while the filtered liquid accumulates on the plate (9) in order then to enter the chimney (10) via the lateral apertures (23) situated in the lower part thereof. Gas and filtered liquid are mixed in the chimneys (10) then emerge, via orifices (18), onto a bed of inert beads (11) situated downstream before reaching the catalytic bed (12) (FIG. 2).

The purpose of this bed of inert beads (11) is to divide the charge stock (C) and redistribute it in the direction of the catalytic bed (12).

On leaving the catalytic bed (12), the fluids (C) pass through a further bed of inert beads (11) (usually organized in an increasing particle size gradient) then a suction strainer or outlet manifold (13) before being removed from the reactor via an outlet (14).

By way of nonlimiting example, the reaction carried out inside such a reactor (1) may be a hydrodesulfuration reaction. The charge fluids (C) (2) are then made up of hydrogen gas H₂ and of liquid hydrocarbons. On leaving the reactor (14), the fluids consist of a desulfured liquid charge, of H₂S and H₂ gas.

One embodiment of the device according to the invention is now described in greater detail with reference to FIGS. 2 to 4.

FIG. 2 depicts a filtration and predistribution device (4) according to the invention, comprising:
a substantially horizontal flat base plate (5) perforated with orifices (16) and which may also be known as a predistribution plate, each orifice in the plate being vertically aligned below a chimney (6), and
a filtration bed (7) made up of filtering elements (8) surrounding the hollow chimneys (6) which are supported by the flat plate (5).

The perforated flat plate (5) also serves to support the filtration bed (7) surrounding each of the chimneys (6).

Said perforated flat plate (5) rests on support beams of the reactor (these have not been depicted) and in terms of its shape and dimensions matches the internal cross section of the reactor (1).

A substantially vertical chimney (6), directed toward the roof of the reactor and assembled with an orifice (16) by means of a cuff (20), is associated with each orifice (16) of the predistribution plate (5).

The external dimensions of the cross sections of the cuffs (20) are chosen to correspond to the orifices (16) in the perforated predistribution plate (5).

With preference, these orifices (16) pass through the entire thickness of the perforated predistribution plate (5) and are identical to one another in terms of shape and dimensions.

Each of the chimneys (6) comprises an upper end that may be covered by some arbitrary capping element (15) and at its periphery comprises at least one aperture (22) passing through its side wall intended to allow the fluids (C) to pass. In the example depicted, each aperture runs in a substantially helical path along the side wall of the chimney, over the entire height of the chimney.

The capping element (15) needs, by virtue of its shape and its dimensions, to allow the reactive gas to pass while at the same time preventing the liquid from entering the chimney (6) via the upper end thereof. Likewise, because of the presence of the capping element (15), the filtering elements (8) that constitute the filtration bed (7) must not be able to enter the chimney (6) when they are being loaded into the reactor.

Thus, the capping element (15) may, for example, be in the form of an inverted cup and be attached by any suitable means (a push fit, clip fastening, welding, etc.) onto a cuff (20) advantageously identical to the one positioned at the lower end of the chimney (6).

The opening of the chimney at its lower end on an orifice (16) of the predistribution plate (5) is intended to allow the fluids to drain toward the distributor plate (9).

Each of the chimneys (6) of the device depicted in the figures is, on the one hand, covered at its upper end by a capping element (15) assembled on a cuff (20) itself mounted on the upper end of the chimney and, on the other hand, associated at its lower end with an orifice (16) in the perforated flat plate (5), by way of a second cuff (20).

The chimneys (6) of the perforated flat plate (5), just like the cuffs (20) and the orifices (16), may be any shape, but are preferably cylindrical.

For preference, as depicted in the example, just one type of cuff (20) is used for mounting the chimney on the orifice (16) and for mounting the capping element (15) on the same chimney.

In the example, the chimney has a cylindrical shape and the cuff is formed of a cylinder with an outside diameter substantially equal to the inside diameter of the chimney.

The cuff (20) can thus be inserted into each end of the chimney, the ends of the chimney and the cuff being shaped to allow manual and reversible assembly of the cuff on these ends.

This highly specific configuration of the ends of the chimneys gives the device (4) a real advantage by making the chimneys removable in a simple and economical way. Having no welds to the perforated predistribution plate (5) but simply being push-fitted onto the latter via the cuffs, the chimneys (6) can therefore readily be removed and exchanged.

Thanks to the removable nature of the chimneys, it is possible for chimneys (6) the geometry of which is dependent on the nature of the filtering elements and other associated elements that are to be loaded onto the perforated predistribution plate (5) around the chimneys (6) to be fitted to the perforated predistribution plate (5) according to what liquid is to be treated. When the chimney is obtained by winding a wire (F′) into turns, the pitch of the turn, and the other parameters of the chimney, will be chosen accordingly.

FIG. 3 depicts an exemplary embodiment of a chimney (6) of the device according to the invention.

In the example depicted in FIG. 3, the chimney (6) is obtained by winding a wire (F′) of cross section (s′) in non-contiguous turns of constant pitch over the entire height. A continuous lateral aperture (22) is thus formed by the space between the turns of the wire that make up the chimney.

The chimney (6) is in the shape of a cylinder the open ends of which each terminate in a radial return (21), or lug, of a length (L) comprised between ⅓ and ⅔ of the diameter (Di′) of the cylinder, as depicted in FIG. 4.

Each end of the chimney may then be associated manually and reversibly by push-fitting (clipping) onto a cylindrical cuff (20) provided with a notch (25) the geometry of which permits assembly of the male/female type with the radial return (21).

In the example depicted in the figures, this notch (25) runs in a vertical plane across a diameter of the cuff and is able to accept the radial return (21) of each end of the chimney.

Such a cuff (20) may then be used, on the one hand, for attaching one of the ends of the chimney to the perforated flat plate (5), the cuff itself being inserted into a circular orifice (16) in the flat plate (5) and, on the other hand, for closing off the other end of the chimney, the second cuff then comprising a capping element (15).

The first cuff is, for example, secured to the perforated flat plate by any suitable means, for example by welding, screwing, bonding, clipping or the like. Likewise, the capping element (15) is attached to the second cuff by welding, bonding, screwing, clipping or the like.

The parameters defining this chimney (6) are as follows:
shape and overall height (H) of the chimney
inside diameter (Di′)
configuration of the spiral aperture (22) and distribution of the pitches of the turns of the wire (F′)
material of the wire (F′)
cross section (s′) of the wire (F′)
dimension of the cross section (s′) of the wire (F′)
length (L) of the radial return or lug (21).

Through its filtration and predistribution action upstream of the catalytic bed, the device (4) of the invention has numerous advantages:
the chimneys (6) and the filtering elements (8) can be inserted on the perforated flat plate (5) simply and inexpensively, especially when the chimneys (6) are removable, particularly when they are assembled by hand using the cuffs (20) with no welding operation, and the filtering elements (8) can be tipped out loose by an operator. It should be noted that the perforated plate (5) not equipped with its chimneys (6) can act as a conventional holed splitter plate;
the position of the device (4) far upstream in the reactor allows the integrity of the volume of the catalytic bed (12) to be preserved and thus also makes it possible to maintain maximum catalyst reactivity;
filtering the fluid makes it possible to reduce the fouling of the mixing chimneys (10) situated on the distributor plate (9), to limit the increase in pressure drop and to guarantee better mechanical integrity of the inert bodies (11);
filtering the liquid fluid prevents fouling of the first layer of the catalytic bed (12). As a result, the descaling of the bed and the costly operations of removing the distribution plate (9) are no longer needed.
the duration of the operating cycle of the reactors (1), which are sensitive to the fouling of the upper layers of the catalytic beds (12), is increased;
the operations of descaling and of changing all or part of the catalyst are limited, thus reducing the costs of interventions.
charges (C) of lower purity can be treated.

The filtering elements (8) that form the filtration bed (7) of the device are now described in greater detail with reference to FIGS. 5 to 8.

These filtering elements (8), which are placed on the perforated flat predistribution plate (5) around the chimneys (6), are hollow elements arranged in the manner of a spring with contiguous and non-contiguous turns and one end of which is closed.

The side view in FIGS. 5 to 8 shows each element in its entirety and, more specifically, the cylindrical or spherical geometry. The plan view gives access to the open and closed ends of the elements and to the nonlimiting variations.

The filtering elements depicted in these figures are obtained by winding a single wire F of cross section (s) in turns. Each element has two ends F1, F2 situated opposite one another along the axis of winding of the turns.

FIG. 5 depicts an element A: this element is cylindrical, with non-contiguous turns of pitch PA and has one open end F1 and one closed end F2 obtained by conical narrowing, of the contiguous-turns type, of the main geometry.

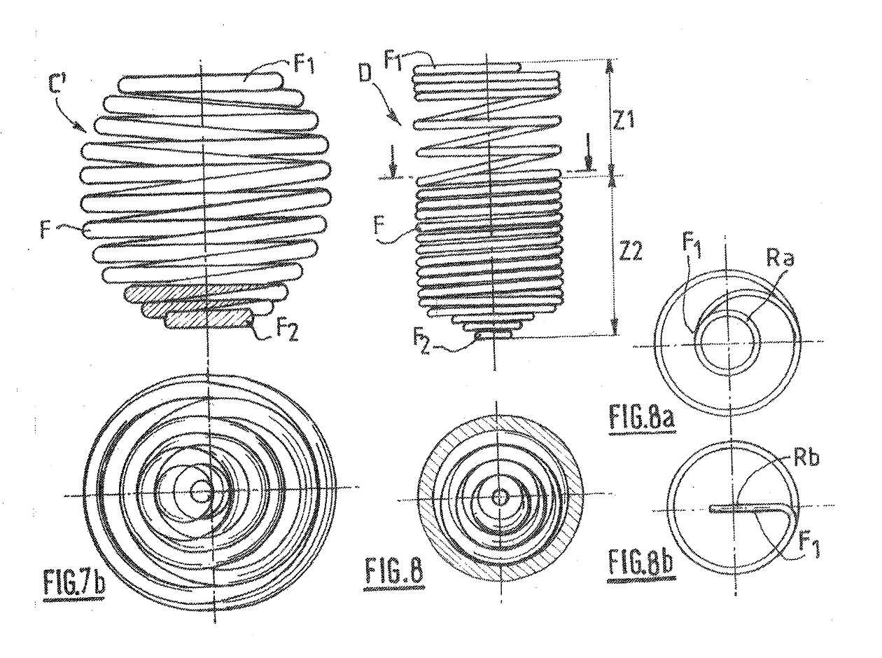

FIG. 6 depicts an element B: this element is spherical, with contiguous turns of pitch PB and has two closed ends F1 and F2.

FIG. 7a depicts an element C: this element is spherical, with contiguous turns of pitch PC and has one open end F1 and one closed end F2.

FIG. 7b depicts an element C′: this element is also spherical, but with non-contiguous turns of pitch PC′, and as a result the geometry of the element is no longer a perfect sphere but a sphere elongated in the direction of the axis of winding of the turns. It too has one open end F1 and one closed end F2.

FIG. 8 depicts an element D: this element is cylindrical, with non-contiguous turns of pitch PD1 in the fluid inlet zone Z1 and PD2 in the fluid filtration zone Z2. The element has one open end F1 associated with Z1 and one closed end F2 associated with Z2 and obtained by conical narrowing, with contiguous turns, of the main geometry. In the variations depicted in FIGS. 8a and 8b, the open end F1 contains a return Ra of the wire of cross section (s) in a concentric circle (FIG. 8a) or else a radial return Rb (FIG. 8b) the length of which is preferably comprised between ⅓ and ⅔ of the diameter of the cylinder. Version D corresponds to the optimum version adopted for performing the filtration tests the results of which are given in the examples.

It should be noted that these filtering elements may differ from one another (from one version to another or within one and the same category) through variations in one or more parameters:
total height of the element
surface area (S_{wire}) occupied by the wire and free surface area (S_{free}) of the element, within the previously-defined ratio S_{free}/S_{wire};
open or closed configuration of the ends and associated geometries;
inside diameter Di of the element;
contiguous or non-contiguous configurations of the turns (pitch) and how they are distributed over the entire height of the element;
material of the wire and geometry, dimensions of its cross section (s);
density of the element.

As described before, the filtering elements (8) may, depending on the fluid to be treated, differ from one another through variations in one or more parameters. Table 1 collates the preferred parameters of the cylindrical and spherical geometries of the filtering elements that make up the filtration bed (7). Tests of loading the filtering elements in bulk onto the perforated plate (5) from the top of the reactor show that the cylindrical geometry is the geometry best suited to obtaining an effective filtration bed. This is because however they are positioned after charging, the cylindrical filtering elements (8) always have openings, thus encouraging good circulation of the fluid and therefore filtration thereof. Once blocked by the buildup of particles, the filtering elements (8) continue to be active, providing the homogeneous dispersion of the purified fluid, a role customarily performed by the inert beads (11). Finally, when the gaps between the filtering elements have themselves become blocked, it is easy for the elements to be removed, cleaned or replaced, the cost of manufacture of which elements is low.

The filtering elements (8) according to the invention as set out in Table 1 each have one closed end and one open end.

TABLE 1

| Element geometry | Cylinder | Sphere |
|---|---|---|
| Dimensions of the | Height (H) ≦50 mm, | Maximum inside diameter |
| element | preferably comprised | (Di) of the sphere ≦50 mm, |
| | between 10 and 35 mm with | preferably comprised |
| | an inside diameter | between 10 and 35 mm. |
| | comprised between 10 and | |
| | 20 mm. | |
| Open end | Simple opening equal to the | Circular opening of |
| | diameter of the cylinder or | diameter ≦ Di/2 |
| | opening terminating in a | |
| | return of the wire in a | |
| | concentric circle of inside | |
| | diameter ranging from 5 to | |
| | 10 mm, or | |
| | opening terminating in a | |
| | radial return of the wire the | |
| | length of which is comprised | |
| | between ⅓ and ⅔ of the | |
| | diameter of the cylinder. | |
| Configuration and | Non-contiguous with pitch ≦1 mm | Non-contiguous turns of |
| distribution of the | over the entire | pitch ≦1 mm, preferably |
| turns | height, preferably ≦0.5 mm, | comprised between 0.2 and |
| | more preferably still, ≦0.2 mm, or | 0.8 mm. |
| | combination of non- | |
| | contiguous turns of pitch ≦1 mm, | |
| | of non-contiguous turns | |
| | of pitch ≦5 mm and of | |
| | 2 to 5 contiguous turns | |
| Closed end | Conical winding on | Closed end of the sphere |
| (geometry, height) | contiguous turns 2 mm to | |
| | 5 mm high. | |
| Ratio S_{free}/S_{wire} | 20% to 25% | 15% to 25% |
| Wire (F) used to | Wire of circular cross | Wire of circular cross |
| make the element | section (s) 0.5 to 1 mm in | section (s) 0.5 to 1 mm in |
| (cross section, | diameter made of steel or | diameter made of steel or |
| material, diameter) | stainless steel and | stainless steel and |
| | preferably of Inox 321 or | preferably of Inox 321 or |
| | 304 | 304 |
| Weight of element | 0.5 to 1 g | 2 to 2.5 g |
| (in grams) | | |
| Density of element | 1.45 to 1.65 | 1.400 |

FIGS. 9 to 19 show the various geometries of the inert bodies tested in the example by comparison with the filtering element of optimum geometry according to version D. These inert bodies are spherical or cylindrical, solid or penetrated by channels of circular, oval or triangular cross section, with or without roughnesses on the surface.

### Examples

The examples set out hereinafter are aimed at illustrating the advantages of the invention.

The Applicant Company has set itself the task of evaluating and comparing the effectiveness of a filtration bed forming part of the filtration device according to the invention.

The prior art shows that, in reactors, the elements used to improve the diffusion of the fluids in order to avoid the creation of preferred paths, which are the causes of hot spots and coking in the catalytic bed, can be termed “filtering” elements. Solid inert beads made of silica and alumina and placed above the catalytic bed are one illustrative example of this. As the liquid fluid circulates it would seem that the solid particles contained therein may build up in the empty gap zones situated between the beads. As the tests will show, this retention of particles on the inert bodies cannot be qualified as “filtration” within the meaning of the invention, inasmuch as it is a result of how the beads are arranged in the reactor rather than being the result of their inherent geometries. The same is true of solid or hollow similar elements based on ceramic, calcium carbonate, quartz or glass. These elements may come with various geometries such as, for example, solid cylinders, four-spoke or seven-spoke wheels, star-like cylinders, spheres with 1 or 5 ducts through them, prisms, etc. Their dimensions may range from a few millimeters to almost 100 mm. Just as was the case with the inert beads, the impurities contained in the charge can accumulate in the empty spaces or be held on the surface roughnesses of the elements. These elements can also be used in applications other than fixed bed catalytic reactors, for example in high-temperature filtration facilities aimed at separating solid and/or liquid particles from the hot gases. Even though they are sometimes qualified as “filtering” elements, these various elements are not filtering within the meaning of the invention because their retention capabilities are dependent on how the elements are arranged relative to one another rather than being dependent on their own inherent geometries. The retention of particles by these elements therefore remains low and uncertain. The tests set out here are aimed at demonstrating this feature by measuring the filtration capability of various filtration beds.

The tests were performed on 13 references of comparative elements customarily used for filtration (cf. FIGS. 9 to 19) as compared against an element D of the filtration bed of the device according to the invention, formed of a hollow cylindrical helical winding closed at one end and open at the other (cf. FIG. 8—version D with a simple opening). With the exception of reference No 4, no other reference has any catalytic activity.

References 1 and 2 are solid spheres with a diameter of 12.7 mm (½″) and 3.175 mm (⅛″) respectively.

References 3 to 13 correspond to the elements depicted in FIGS. 9 to 19 respectively.

The filtering element of the filtration bed of the device according to the invention (element D) used in these tests is defined by the following parameters:
Total height of the element: 23 mm
22%≦S_{free}/S_{wire}≦23%
P1/P2≦5
Inside diameter Di of the element: 10 mm
Configuration of the element:

Cylinder 20 mm high consisting of one open end followed by 3 contiguous turns, themselves followed by a zone Z1 made up of 2 non-contiguous turns with a constant pitch PD1 of 3 mm, said zone Z1 being followed by a zone Z2 made up of non-contiguous turns with a constant pitch PD2 of 1 mm over a height of 8 mm, said zone Z2 being followed by a conical closed end with contiguous turns over a height of 3 mm.
Wire of Inox 321 of circular cross section 0.8 mm in diameter. The tests involved evaluating the retention power of a filtration bed consisting of a certain reference of filtering elements. The elements of one and the same reference were thus loaded in loose to form a filtration bed on a column 60 cm high and 10 cm in diameter.

In the first series of tests, each of the beds consisting of one of the 14 references was weighed empty then subjected, for 2 hours, to a liquid flow rate (120 l/h of water) laden with clogging particles (2 kg of solid particles with a particle size varying from 10 μm to 400 μm) and to a gas flow rate (2.5 m³/h of air). At the end of each test, the particle-laden elements that constituted the filtration beds were dried in an oven at 120° C. for 24 hours and then weighed. Table 2 collates the results of this first series and reveals the overall filtration capability of a filtration bed consisting of one same category of elements.

These tests demonstrate the very low filtration capability of the majority of the elements tested: 86% of the filtration beds retain under 3% of particles. The two best-performing filtration beds respectively retain a little over 7% of particles in the case of the filtration bed made up of the elements bearing the reference No. 12 and a little over 5% in the case of the filtration bed consisting of the elements bearing the reference No. 14 (filtering element D according to the invention).

Studying with the naked eye the elements D of the filtration bed of the device according to the invention shows that the particles accumulate on the inside of the elements until these elements become saturated.

In the second series of tests, the two filtration beds consisting of the best-performing filtration elements (references 12 and 14) determined in series 1 of tests were subjected continuously to 3 successive passes, each lasting 2 hours, of the liquid laden with clogging particles at a gas flow rate (namely 3 times 120 l/h of water laden with 2 kg of solid particles with a particle size varying from 10 to 400 μm under an air flow rate of 2.5 m³/h). Between each pass, the elements under test were neither cleaned nor replaced. The particle-laden elements that made up the filtration beds were weighed after drying in an oven (120° C. for 24 hours). These cumulative tests show that the hollow elements formed of a helical winding according to the invention have almost twice the filtration capability of the elements No. 12. These elements therefore perform an “active” filtration resulting from their own inherent geometry, unlike the elements No. 12 which become saturated more quickly.

The results of these cumulative tests (Table 2) show that, thanks to their suitable geometry, the elements of the filtration bed of the device according to the invention actively filter the particle-laden liquid, these particles accumulating within the elements until they entirely fill them.

Elements No. 12 perform only “passive retention” of the particles which accumulate in the gaps left free between the elements. The roughnesses on the surface of the elements No. 12 are able to capture particles, but rapidly become saturated and do not allow the capture of a significant volume of particles.

Unlike the hollow elements formed of a helical winding, the other elements lack effectiveness and cannot therefore be qualified as filtering within the meaning of the invention.

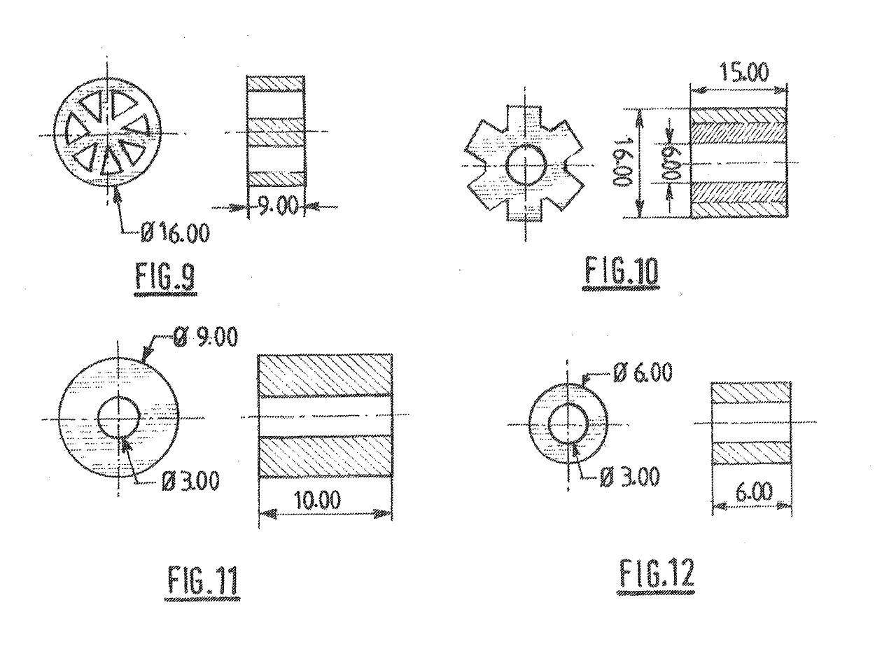

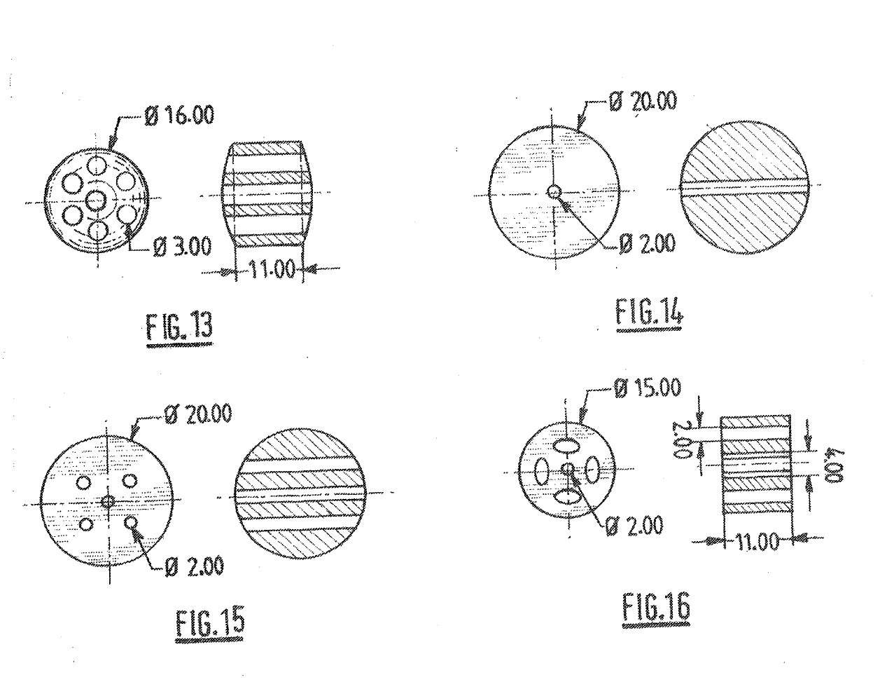

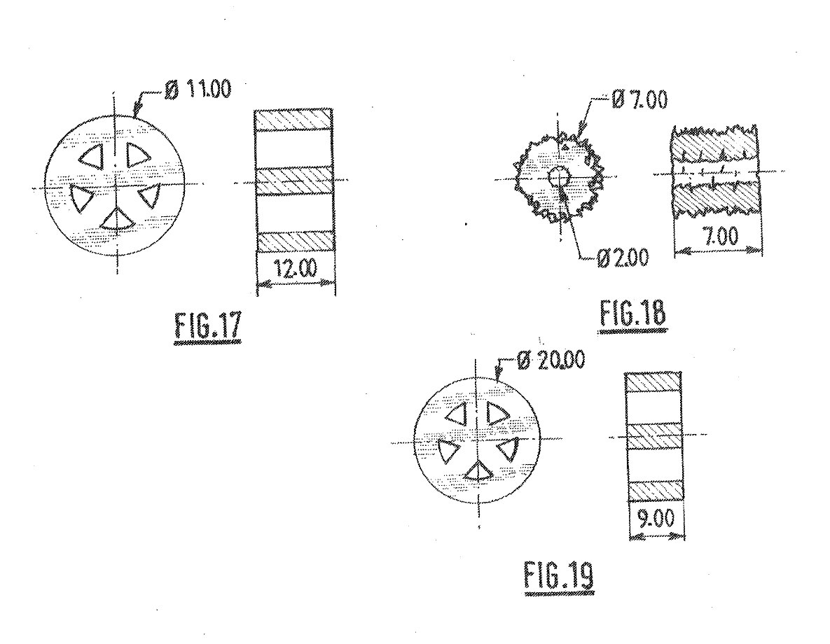

TABLE 2

| | | | Series 1 | | | |
|---|---|---|---|---|---|---|
| | | | (2 h) | | Series 2 | |
| | | | | % | Cumulative | |
| | | | | retained by | tests (3 × 2 h) | |
| | | | | the column | without cleaning of | |
| | | | Mean | with respect | the column | |
| | | | total mass | to the | Mean total | |
| Reference | | | retained | quantity (by | mass | % |
| No. | | | by the | weight) of | retained by | retained |
| (associated | | Dimensions | column | particles | the column | by the |
| figure) | Geometry | (mm) | (grams) | introduced | (grams) | column |
| 1 | Sphere | 12.7 | 6 | 0.3 | | |
| | (beads) | (½″) | | | | |
| 2 | Sphere | 3.175 | 37 | 1.9 | | |
| | (beads) | (⅛″) | | | | |
| 3 | 7-spoke | 16 × 9 | 44 | 2.21 | | |
| (FIG. 9) | wheel | | | | | |
| 4 | Star-like | 15 × 16 | 14 | 0.70 | | |
| (FIG. 10) | cylinder | | | | | |
| 5 | Hollow | 9 × 10 | 41 | 2.1 | | |
| (FIG. 11) | cylinder | | | | | |
| 6 | Hollow | 6 × 6 | 30 | 1.8 | | |
| (FIG. 12) | cylinder | | | | | |
| 7 | Wheel | 16 × 11 | 40 | 2.0 | | |
| (FIG. 13) | pierced with | | | | | |
| | 7 holes | | | | | |
| 8 | Macroporous | 20 | 8 | 0.4 | | |
| (FIG. 14) | sphere 1 hole | | | | | |
| 9 | Macroporous | 20 | 8 | 0.4 | | |
| (FIG. 15) | sphere 5 | | | | | |
| | holes | | | | | |
| 10 | Pierced 4- | 15 × 11 | 48 | 2 | | |
| (FIG. 16) | spoke wheel | | | | | |
| | (5 holes) | | | | | |
| 11 | “Pentaring” | 11 × 12 | 48 | 2.4 | | |
| (FIG. 17) | | | | | | |
| 12 | Granular | 7 × 7 | 145 | 7.2 | 98 | 4.9 |
| (FIG. 18) | hollow | (D_{hollow} = 2) | | | | |
| | cylinder | | | | | |
| 13 | “Pentaring” | 20 × 9 | 35 | 1.7 | | |
| (FIG. 19) | | | | | | |
| 14 | Cylindrical | Inside | 105 | 5.25 | 160 | 8.1 |
| (FIG. 8) | element D | diameter 10 | | | | |
| | with helical | length 10 | | | | |
| | winding | | | | | |

1. A device for the filtration and predistribution (4) of at least one particle-laden fluid, characterized in that said device comprises:
a flat plate (5) perforated with orifices (16), each orifice of the plate lying in vertical alignment below a vertical hollow chimney (6) comprising at least one aperture passing through its side wall,
a filtration bed (7) arranged on the perforated plate and surrounding said chimneys, the filtration bed comprising at least one layer of hollow filtering elements (8) the dimensions of which are greater than the dimensions of the apertures of the chimneys, said filtering element being obtained by winding a wire of cross section (s) into contiguous and/or non-contiguous turns so as to have at least one closed end, and having a ratio of the free surface area (S_{free}) of the element to the surface area (S_{wire}) occupied by the wire that is comprised between 2 and 50%.

2. The device for filtration and predistribution as claimed in claim 1, characterized in that each chimney of the perforated plate (5) is removable.

3. The device for filtration and predistribution as claimed in claim 1, characterized in that said at least one aperture (22) of each chimney extends in a substantially helical path along the side wall of the chimney.

4. The device for filtration and predistribution as claimed in claim 1, characterized in that the density with which the orifices (16) are distributed over the perforated flat plate (5) is comprised between 5 and 150 orifices per m² of plate surface area, preferably between 30 and 100 orifices per m² of plate surface area.

5. The device for filtration and predistribution as claimed in claim 1, characterized in that each chimney (6) is obtained by winding a wire of cross section (s′) into non-contiguous turns of constant pitch over its entire height.

6. The device for filtration and predistribution as claimed in claim 1, characterized in that each chimney (6) is in the form of a cylinder and has open ends at least one of which terminates in a radial return of the wire of cross section (s′) of a length comprised between ⅓ and ⅔ of the diameter of the chimney.

7. The device for filtration and predistribution as claimed in claim 1, characterized in that the height of each chimney (6) is comprised between 100 and 1500 mm, preferably between 150 and 600 mm.

8. The device for filtration and predistribution as claimed in claim 1, characterized in that at least one end of the chimney is shaped so that it can be fitted by hand and reversibly onto a cylindrical cuff (20).

9. The device for filtration and predistribution as claimed in claim 8, characterized in that one end of the chimney is provided with a cuff inserted in an orifice (16) in the perforated plate (5), and the other end of the chimney (6) is provided with a cuff (20) covered by a capping element (15).

10. The device for filtration and predistribution as claimed in claim 1, characterized in that each chimney (6) extends beyond the level of the filtration bed (7) by a height comprised between 20 and 70 mm, preferably between 30 and 60 mm.

11. The device for filtration and predistribution as claimed in claim 1, characterized in that the ratio S_{free}/S_{wire} is comprised between 5 and 30%, preferably between 15 and 25%.

12. The device for filtration and predistribution as claimed in claim 1, characterized in that the filtering element (8) is in the shape of a cylinder or in the shape of a sphere.

13. The device for filtration and predistribution as claimed in claim 1, characterized in that the filtering element (8) comprises an open end followed by a fluid inlet zone Z1 consisting of non-contiguous turns of pitch P1, followed by a filtration zone Z2 consisting of non-contiguous turns of pitch P2<P1 and which is extended by a closed end.

14. The device for filtration and predistribution as claimed in claim 1, characterized in that the filtering element (8) is made of a metallic material, preferably steel or stainless steel.

15. The device for filtration and predistribution as claimed in claim 1, characterized in that the filtering elements (8) of one and the same layer of the filter bed (7) are identical to one another.

16. The device for filtration and predistribution as claimed in claim 1, characterized in that the filter bed (7) consists of several layers organized in a gradient graded on filtering element size.

17. The device for filtration and predistribution as claimed in claim 1, characterized in that the filtering elements (8) of one and the same layer of the filter bed (7) are used alone or in combination with other elements.

18. The device for filtration and predistribution as claimed in claim 17, characterized in that the combined elements are particles of catalyst and/or inert beads.

19. The use of the device (4) for filtration and predistribution as claimed in claim 1 in a reactor (1) comprising at least one fixed catalytic bed (12), the reactor being fed with at least one particle-laden liquid and one reactive gas, said device (4) being situated upstream of the catalytic bed (12), the flat plate (5) being parallel to the transverse cross section of the reactor (1).

20. The use of the device (4) for filtration and predistribution as claimed in claim 19, characterized in that the device is situated upstream of a distributor plate (9) itself situated upstream of the fixed catalytic bed (12).

21. The use of the device (4) for filtration and predistribution as claimed in claim 1 for reactions of hydrotreatment, selective hydrogenation, or conversion of hydrocarbon-containing residues or cuts.
